(19) United States
(12) Patent Application Publication
(10) Pub. No.: US 2017/0319443 A1
(43) Pub. Date: Nov. 9, 2017

(54) MULTIPLE EMULSIONS COMPRISING RIGIDIFIED PORTIONS

(71) Applicants: President and Fellows of Harvard College, Cambridge, MA (US); The Procter & Gamble Company, Cincinnati, OH (US)

(72) Inventors: David A. Weitz, Bolton, MA (US); Alireza Abbaspourrad, Ithaca, NY (US); Chang-Hyung Choi, Belmont, MA (US); Hyomin Lee, Cambridge, MA (US); Marco Caggioni, Cincinnati, OH (US); John Christopher, Liberty Township, OH (US); Taotao Zhu, West Chester, OH (US)

(73) Assignees: President and Fellows of Harvard College, Cambridge, MA (US); The Procter & Gamble Company, Cincinnati, OH (US)

(21) Appl. No.: 15/528,905

(22) PCT Filed: Nov. 19, 2015
(86) PCT No.: PCT/US2015/061511

§ 371 Date: May 23, 2017

Related U.S. Application Data
(60) Provisional application No. 62/083,721, filed on Nov. 24, 2014.

(51) Int. Cl. A61K 8/06; A23P 10/30; B01J 13/10; A61K 8/11; A61K 8/81; B01F 17/00; B01F 13/00; B01F 5/04; B01F 3/08; A61Q 13/00; A61K 9/113; A61K 8/86; B01J 13/16

(52) U.S. Cl. CPC A61K 8/066; B01J 13/16; A23P 10/30; B01J 13/10; A61K 8/11; A61K 8/8129; B01F 17/0028; B01F 13/0062; B01F 5/045; B01F 3/0807; A61Q 13/00; A61K 9/113; A61K 8/86; A61K 8/8152; A61K 2800/624; A61K 2800/413; B01F 2003/0838

(57) ABSTRACT

The present invention generally relates to microfluidic droplets and, in particular, to multiple emulsion microfluidic droplets. In one set of embodiments, multiple emulsion droplets are provided, where an inner shell of the droplet is relatively thin, compared to the outer shell (or other shells) of the droplet. For instance, in one set of embodiments, the inner droplet has an average thickness of less than about 1000 nm. In some cases, the inner shell may be rigidified, e.g., to form a gel or a polymeric layer. This may be useful, for example, for preventing coalescence of fluids within the microfluidic droplet. Other embodiments of the present invention are generally directed to methods of making such droplets, methods of using such droplets, microfluidic devices for making such droplets, and the like.

### RELATED APPLICATIONS

This application claims the benefit of U.S. Provisional Patent Application Ser. No. 62/083,721, filed Nov. 24, 2014, entitled “Systems and Methods for Encapsulation of Actives in Compartments or Sub-Compartments,” by Weitz, et al., incorporated herein by reference.

### FIELD

The present invention generally relates to microfluidic droplets and, in particular, to multiple emulsion microfluidic droplets.

### BACKGROUND

Double emulsions are drops containing at least one smaller drop that is composed of a second, substantially immiscible fluid. These core-shell structured fluids can be used, for instance, as templates to produce capsules; the outer drop contains the material that ultimately forms the shell of the capsule, whereas the inner drop constitutes the capsule interior core. These capsules can be used as vehicles for delivery of active ingredients in many fields, such as food, pharmaceuticals, or cosmetics. However, successful application of these capsules may require good control over their permeability and mechanical stability, parameters that can be tuned with the composition and thickness of the capsule shell. This may involve control over the dimensions and composition of the double emulsions. This control is often difficult to achieve if double emulsions are produced by mechanical stirring or membrane emulsification, since these conventional approaches typically yield double emulsion drops of different sizes that often contain multiple inner droplets.

### SUMMARY

The present invention generally relates to microfluidic droplets and, in particular, to multiple emulsion microfluidic droplets. The subject matter of the present invention involves, in some cases, interrelated products, alternative solutions to a particular problem, and/or a plurality of different uses of one or more systems and/or articles.

In one aspect, the present invention is generally directed to a composition. According to one set of embodiments, the composition comprises a first droplet comprising a first fluid, where the first droplet is contained within a second droplet comprising a second fluid, where the second droplet is contained within a third droplet comprising a third fluid. In some cases, the second droplet has an average thickness, between the first droplet and the third droplet, of less than about 1000 nm or less than about 100 nm.

In another set of embodiments, the composition comprises a first droplet comprising a first fluid, where the first droplet is contained within a second droplet comprising a second fluid, where the second droplet is contained within a third droplet comprising a third fluid. In certain cases, the second fluid comprises less than about 10% of the volume of the third droplet, and/or the first fluid comprises at least about 50% of the volume of the third droplet.

The composition, in yet another set of embodiments, comprises a first droplet comprising a first fluid, where the inner droplet is contained within a second droplet comprising a second fluid, where the second droplet is contained within a third droplet comprising a third fluid. In some embodiments, the difference between the average diameter of the second droplet and the average diameter of the first droplet is less than about 10% of the average diameter of the third droplet.

In another aspect, the present invention is generally directed to a method. In some cases, the method is a method for forming any of the droplets discussed herein, including those discussed above.

The method, in one set of embodiments, includes flowing a first fluid in a first microfluidic conduit, expelling the first fluid from an exit opening of the first conduit into a second fluid in a second microfluidic conduit such that droplets of first fluid are formed at the exit opening of the first conduit, and expelling the droplets of first fluid contained within the second fluid from an exit opening of the second conduit into a third fluid contained within a third microfluidic conduit. In some embodiments, the method is used to form a multiple emulsion droplet comprising a first droplet containing the first fluid, surrounded by a second droplet containing the second fluid, surrounded by a third droplet containing the third fluid, wherein the second droplet has an average thickness, between the first droplet and the third droplet, of less than about 1000 nm or less than about 100 nm.

In another set of embodiments, the method may include flowing a first fluid in a first microfluidic conduit, expelling the first fluid from an exit opening of the first conduit into a second fluid in a second microfluidic conduit such that droplets of first fluid are formed at the exit opening of the first conduit, and expelling the droplets of first fluid contained within the second fluid from an exit opening of the second conduit into a third fluid contained within a third microfluidic conduit. In some embodiments, the method is used to form a multiple emulsion droplet comprising a first droplet containing the first fluid, surrounded by a second droplet containing the second fluid, surrounded by a third droplet containing the third fluid. In certain cases, the middle fluid comprises less than about 10% of the volume of the outer droplet, and/or the inner fluid comprises at least about 50% of the volume of the outer droplet.

In another aspect, the present invention encompasses methods of making one or more of the embodiments described herein, for example, microfluidic droplets containing actives or other species. In still another aspect, the present invention encompasses methods of using one or more of the embodiments described herein, for example, microfluidic droplets containing actives or other species.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A and 1B illustrate methods of forming triple emulsion droplets, in accordance with one set of embodiments;

FIGS. 2A-2C illustrate certain triple emulsion droplets and methods of making such droplets, in another set of embodiments;

FIGS. 3A-3G illustrate certain triple emulsion droplets, in accordance with yet another set of embodiments;

FIGS. 4A-4C illustrate certain triple emulsion droplets having a rigidifying layer, in another set of embodiments;

FIGS. 5A-5B illustrate separated droplets of ETPTA and oil, in certain embodiments; and

FIGS. 6A-6B illustrate ruptured droplets, in still another set of embodiments.

### DETAILED DESCRIPTION

The present invention generally relates to microfluidic droplets and, in particular, to multiple emulsion microfluidic droplets. In one set of embodiments, multiple emulsion droplets are provided, where an inner shell of the droplet is relatively thin, compared to the outer shell (or other shells) of the droplet. For instance, in one set of embodiments, the inner droplet has an average thickness of less than about 1000 nm. In some cases, the inner shell may be rigidified, e.g., to form a gel or a polymeric layer. This may be useful, for example, for preventing coalescence of fluids within the microfluidic droplet. Other embodiments of the present invention are generally directed to methods of making such droplets, methods of using such droplets, microfluidic devices for making such droplets, and the like.

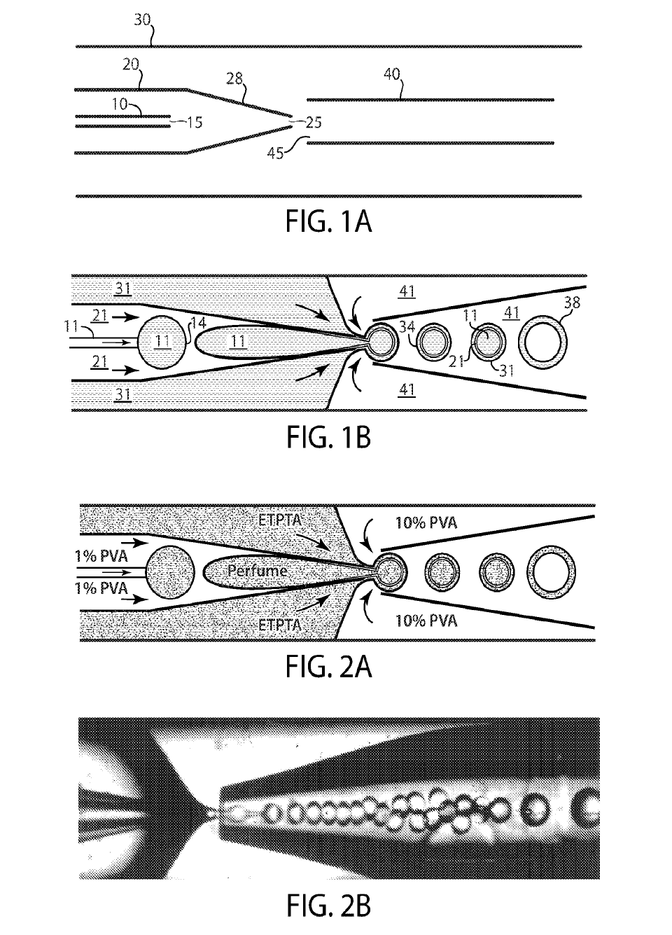

Turning first to FIG. 1, this figure illustrates one example method of producing multiple emulsion microfluidic droplets. In FIG. 1A, microfluidic device 5 is shown. Device 5 includes a first conduit 10, which contains an exit opening 15 leading into second conduit 20. Second conduit 20 contains a tapered portion 28 which leads into an exit opening 25. Exit opening 25 is contained within a third conduit 30, and faces an entrance opening 45 to a fourth (or exit) conduit 40.

FIG. 1B shows the flow of fluid within microfluidic device 5. In this particular example, first fluid 11 flows in through conduit 10, exiting through exit opening 15 into a second fluid 21 contained within second conduit 20. First fluid 11 and second fluid 12 may be substantially immiscible, thereby causing first fluid 11 to form discrete droplets 14 within second fluid 12. In some cases, the flowrate of the first fluid is relatively slow, e.g., such that droplets are created in the “dripping regime,” rather than through a “jetting” process.

Droplet 14 (containing first fluid 11), once created, may move towards exit opening 25 of conduit 20. In some cases, conduit 20 may taper towards exit opening 25, thereby causing droplet 14 to become extended towards the exit opening. However, droplet 14 may be prevented from coming into physical contact with the walls of conduit 20 due to the presence of second fluid 21. In some embodiments, second fluid 21 may exhibit greater attraction to the walls of conduit 20 than first fluid 11. For example, this may be an inherent attraction (e.g., if second fluid 21 and the walls of conduit 20 are both hydrophilic or both hydrophobic), or in some cases, the walls of conduit 20 are coated or reacted to render them more attractive to second fluid 21 than first fluid 11. The attraction may accordingly facilitate the production of droplets surrounded by a relatively thin, inner shell of second fluid.

Upon exiting through exit opening 25, droplet 11 is surrounded by second fluid 21. Depending on the shape of the tapered portion of conduit 20, and/or of the flow rates of first fluid 11 and second fluid 21, however, there may be a relatively small amount of second fluid that surrounds droplet 11. Upon exiting through the exit opening, the fluids may come into contact with a third fluid 31 flowing through conduit 30 from left to right, towards entrance opening 45 of conduit 40. Third fluid 31 may be caused to form droplets upon interaction with a fourth fluid 41, flowing from right to left within conduit 30. Upon exiting through exit opening 45, fourth fluid 41 may be continuous, containing discrete droplets 34 of third fluid 31. In addition, the droplets may also contain a droplet of second fluid 21, which in turn contains a droplet of first fluid 11. It should be noted that first fluid 11 and third fluid 31 may be miscible or immiscible, since they do not come into direct contact with each other; in some cases, they may even be the same fluid. Similarly, second fluid 21 and fourth fluid 41 may be miscible or immiscible, since they do not come into direct contact with each other, and they may even be the same fluid in certain embodiments. Accordingly, multiple emulsion droplets can be formed, including a droplet of first fluid 11 contained within a droplet of second fluid 21, which is contained in a droplet of third fluid 31, which is contained in a continuous fourth fluid 41. In addition, in some cases, some droplets may form without first fluid 11, e.g., as is shown with droplet 38.

In one set of embodiments, the droplet may contain a relatively thin “shell” of inner fluid 21. In some cases, this may give the appearance of a double emulsion droplet containing two fluids (fluids 11 and 31), contained within carrying fluid 41, although it should be understood that in reality, first fluid 11 and third fluid 31 (which may be miscible in some cases) are not actually touching and do not mix, due to the presence of intervening second fluid 21. In some cases, the second fluid may have a relatively thin average cross-section or thickness, for instance, less than about 1000 nm or less than about 100 nm. In addition, in some cases, the second fluid may be rigidified, e.g., to form a gel or a solid layer. For instance, the second fluid may contain a monomer that can polymerize when exposed to ultraviolet (UV) light in the presence of a photoinitiator. This may be useful, for example, for preventing the first fluid and the third fluid from coming into contact with each other and coalescing or merging together. Thus, for example, after formation of the multiple emulsion droplet, the droplet may be exposed to ultraviolet light from a suitable ultraviolet light source to cause rigidification of the second fluid to occur, e.g., forming a relatively rigid layer between the first and third fluids, thereby preventing them from touching or mixing.

Such embodiments may be useful, for example, in embodiments where a relatively large amount of first fluid 11 is to be encapsulated within third fluid 31, and where second fluid 21 is mostly used to separate first fluid 11 and third fluid 31. In addition, in some cases, one or more of the first or third fluids may also be hardened or rigidified. For example, the third fluid 31 may be hardened or rigidified to create a capsule containing the first fluid.

The above discussion is a non-limiting example of one embodiment of the present invention that can be used to produce droplets or particles having relatively thin inner shells or layers, which in some cases may be rigidified, e.g., to form a gel or a polymer. However, other embodiments are also possible. Accordingly, more generally, various aspects of the invention are directed to various systems and methods for producing triple and other multiple emulsion microfluidic droplets, e.g., having relatively thin inner shells or layers.

In one aspect, the present invention is generally directed to a triple or higher multiple emulsion. Generally, in a triple emulsion, a first (or inner) fluidic droplet comprising a first fluid is surrounded by a second (or middle) fluidic droplet comprising a second fluid, which in turn is surrounded by a third (or outer) fluidic droplet comprising a third fluid, which is contained within a continuous or carrying fourth fluid. Typically, a fluid is substantially immiscible with an adjacent fluid, although fluids that are not adjacent need not be immiscible, and may be miscible (or even identical) in some cases. Thus, for example, the first fluid may be immiscible with the second fluid, but may be miscible or immiscible with the third fluid or the fourth fluid. Similarly, the second fluid may be immiscible with the third fluid, but may be miscible or immiscible with the fourth fluid. However, it should be understood that immiscibility is not necessarily required in all embodiments; in some cases, two adjacent fluids are not immiscible, but may retain separation in other ways, e.g., kinetically or through short exposure times.

Thus, as a non-limiting example, in a triple emulsion droplet, the first fluid (innermost fluid) may be an aqueous or hydrophilic fluid (a “water” phase), the second fluid (middle fluid) may be a lipophilic or hydrophobic or “oil” phase that is substantially immiscible with the aqueous fluid, the third fluid (or outer fluid) may be an aqueous fluid (a “water” fluid) that is substantially immiscible with the second fluid, and the fourth (or carrying) fluid may be a lipophilic or “oil” phase that is substantially immiscible with the third fluid. This is sometimes generally referred to as a W/O/W/O triple emulsion droplet (for water/oil/water/oil), although it should be understand that this is mainly for the sake of convenience; for instance, the first fluid can be any suitable aqueous fluid, and it need not be pure water. For example, the aqueous fluid may be water, saline, an aqueous solution, ethanol, or the like, or any other fluid miscible in water. The oil, in contrast, may be immiscible in water, at least when left undisturbed under ambient conditions. In similar fashion, an O/W/O/W triple emulsion droplet may be similarly defined. Furthermore, these principles may be extended to higher-order multiple emulsions droplets. For example, a quadruple emulsion droplet may comprise a first fluid, surrounded by a second fluid, surrounded by a third fluid, surrounded by a fourth fluid, contained in a fifth fluid, etc. In addition, it should be understood that other arrangements are also possible. For example, in one embodiment, the first fluid, the second fluid, and the third fluid may be all mutually immiscible. Furthermore, some embodiments of the present invention are generally directed to higher multiple emulsions, e.g., quadruple emulsions, quintuple emulsions, etc. One (or more) of the inner shells of the multiple emulsion may be relatively thin, e.g., as discussed herein with respect to the second fluid of a triple emulsion droplet.

As used herein, two fluids are immiscible, or not miscible, with each other when one is not soluble in the other to a level of at least 10% by weight at the temperature and under the conditions at which the emulsion is produced. For instance, two fluids may be selected to be immiscible within the time frame of the formation of the fluidic droplets. In some embodiments, two fluids (e.g., the carrying fluid and the inner droplet fluid of a multiple emulsion) are compatible, or miscible, while the outer droplet fluid is incompatible or immiscible with one or both of the carrying and inner droplet fluids. In other embodiments, however, all three (or more) fluids may be mutually immiscible, and in certain cases, all of the fluids do not all necessarily have to be water soluble. In still other embodiments, as mentioned, additional fourth, fifth, sixth, etc. fluids may be added to produce increasingly complex droplets within droplets, e.g., a carrying fluid may surround a first fluid, which may in turn surround a second fluid, which may in turn surround a third fluid, which in turn surround a fourth fluid, etc. In addition, the physical properties of each nesting layer of fluidic droplets may each be independently controlled, e.g., by control over the composition of each nesting level.

In certain aspects, the second fluid may be relatively thin. For example, the second fluid (or other inner fluid having a relatively thin shell) may have an average thickness (i.e., between the first fluid and the second fluid) of less than about 1 micrometer, less than about 700 nm, less than about 500 nm, less than about 300 nm, less than about 200 nm, less than about 100 nm, less than about 50 nm, less than about 30 nm, less than about 20 nm, or less than about 10 nm. The thickness may be determined optically or visually, or in some cases, estimated based on the volumes and/or flowrates of fluid entering or leaving a conduit. In some cases, the second fluid (or other inner fluid having a relatively thin shell) may have an average thickness of at least about 10 nm, at least about 20 nm, at least about 30 nm, at least about 50 nm, at least about 100 nm, at least about 200 nm, at least about 300 nm, at least about 500 nm, at least about 700 nm, etc. Combinations of any of these are also possible, e.g., the thickness may be between about 300 nm and about 700 nm. If the droplet is non-spherical, then average thickness or diameters may be determined or estimated by using a perfect sphere having the same volume as the non-spherical droplet(s).

The volumes or thicknesses of a layer of fluid in a droplet may be determined or estimated (e.g., before and/or after distortion) using any suitable technique, e.g., visually or optically. In some cases, the volumes or thickness of a layer of fluid may be estimated statistically, e.g., by determining the amount of fluid present in a plurality of double or other multiple emulsion droplets, and assuming that the droplets are spherical, calculating the volume and/or thicknesses of the fluid around each droplet.

In addition, in some embodiments, the thickness may be determined as a percentage of the diameter of the overall droplet within the carrying fluid. For example, the thickness of the second fluid (or other inner fluid having a relatively thin shell) within the droplet may be than about 20%, less than about 15%, less than about 10%, less than about 5%, less than about 3%, less than about 1%, less than about 0.5%, less than about 0.3%, or less than about 0.1% of the diameter of the overall droplet.

In addition, in some embodiments, the second fluid (or other inner fluid having a relatively thin shell) may comprise a relatively small percentage by volume of the overall droplet. For example, the second fluid may comprise less than about 20%, less than about 15%, less than about 10%, less than about 5%, less than about 3%, less than about 1%, less than about 0.5%, less than about 0.3%, or less than about 0.1% of the overall droplet. In another set of embodiments, the second fluid (or other inner fluid having a relatively thin shell) may have a thickness such that the difference between the average diameter of a droplet containing the second fluid and the average diameter of a droplet contained therein is less than about 20% of the average diameter of the overall droplet, and in some cases, less than about 15%, less than about 10%, less than about 5%, less than about 3%, less than about 1%, less than about 0.5%, less than about 0.3%, or less than about 0.1% of the average diameter of the overall droplet.

In some embodiments, the second fluid (or other inner fluid having a relatively thin shell) may have an average thickness of less than about 0.05, less than about 0.01, less than about 0.005, or less than about 0.001 times the average cross-sectional diameter of the droplet, or between about 0.0005 and about 0.05, between about 0.0005 and about 0.01, between about 0.0005 and about 0.005, or between about 0.0005 and about 0.001 times the average cross-sectional diameter of the droplet. In some embodiments, the second fluid (or other inner fluid having a relatively thin shell) of a droplet may have an average thickness of less than about 1 micron, less than about 700 nm, less than about 500 nm, less than about 300 nm, or less than about 100 nm, or between about 50 nm and about 1 micron, between about 50 nm and about 500 nm, between about 300 nm and about 700 nm, or between about 50 nm and about 100 nm. One of ordinary skill in the art would be capable of determining the average thickness, for example, by examining scanning electron microscope (SEM) images of the droplets.

In addition, in some embodiments as discussed herein, the second fluid may be rigidified or hardened. For instance, the droplet may comprise a second droplet that is present as a gel or a polymer. This droplet (or layer) may be used to prevent the first fluid and the third fluid from coming into contact with each other. For example, the gel may be a hydrogel, such as agarose, or a polymer, such as polyacrylamide, poly(N-isopropylacrylamide), or poly(ethylene glycol diacrylate).

It should also be understood that in some cases, the first (or inner) droplet contained within the second droplet is relatively large, e.g., a large percentage of the volume of the second droplet is taken up by the first droplet, which may result in the second droplet having a relatively thin thickness, as discussed above. Thus, for example, on a volume basis, the first droplet may take up at least about 80% of the volume of the second droplet, and in some cases, at least about 85%, at least about 90%, at least about 95%, at least about 97%, at least about 98%, at least about 99%, at least about 99.5%, or at least about 99.7% of the volume of the second droplet. In some cases, the diameter of the first (or inner) droplet may be at least about 80% of the diameter of the second droplet, and in some cases, at least about 85%, at least about 90%, at least about 95%, at least about 97%, at least about 98%, at least about 99%, at least about 99.5%, or at least about 99.7% of the diameter of the second droplet.

In one set of embodiments, the inner fluid comprises at least about 50% of the volume of the overall droplet, and in some cases, at least about 60%, at least about 70%, at least about 75%, at least about 80%, or at least about 85% of the volume of the outer droplet. In some cases, the volume of the inner fluid may also be no more than about 90%, no more than about 85%, no more than about 80%, no more than about 75%, no more than about 70%, no more than about 65%, no more than about 60%, or no more than about 55% of the volume of the overall droplet. Combinations of any of these are also possible, e.g., the inner fluid may comprise between about 50% and about 80% of the volume of the overall droplet.

The droplets may be microfluidic droplets, in some instances. For instance, the outer droplet may have a diameter of less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers, or between about 50 micrometers and about 1 mm, between about 10 micrometers and about 500 micrometers, or between about 50 micrometers and about 100 micrometers in some cases. However, in some cases, the droplets may be larger. For example, the inner droplet (or a middle droplet) of a triple or other multiple emulsion droplet may have a diameter of less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers, or between about 50 micrometers and about 1 mm, between about 10 micrometers and about 500 micrometers, or between about 50 micrometers and about 100 micrometers in some cases.

In some embodiments, by controlling the volumes and/or flow rates of the various fluids, the volumes and/or thicknesses of the components of the triple or other multiple emulsion droplets may be controlled. For instance, in one set of embodiments, triple emulsion droplets are formed such that the droplets contain a relatively large amount of first fluid but a lesser amount of second fluid, i.e., droplets may be formed that have relatively thin “shells” or “layers” of second fluid surrounding the first fluid. As noted herein, the second fluid is rigidified in certain embodiments, e.g., forming a gel or a polymer. Examples of gels or polymers include any of those discussed herein.

In certain aspects, the microparticle may have a relatively thin wall thickness. For example, the average or mean wall thickness may be less than about 10 micrometers, less than about 5 micrometers, less than about 3 micrometers, less than about 1 micrometer, less than about 500 nm, less than about 300 nm, less than about 200 nm, less than about 100 nm, less than about 50 nm, less than about 30 nm, less than about 20 nm, or less than about 10 nm. In some cases, the mean wall thickness may be at least about 0.1 micrometers, at least about 0.3 micrometers, at least about 0.5 micrometers, at least about 1 micrometer, at least about 3 micrometers, at least about 5 micrometers, or at least 10 micrometers. Combinations of any of these are also possible; for instance, the mean wall thickness may be between about 0.1 micrometer and about 10 micrometers. The thickness may be determined optically or visually, or in some cases, estimated based on the volumes and/or flowrates of fluid entering or leaving a conduit. If the microparticle is non-spherical, then average thickness or diameters may be determined using a perfect sphere having the same volume as the non-spherical microparticle.

The volumes or thicknesses of a layer of in a microparticle may be determined or estimated (e.g., before and/or after distortion) using any suitable technique, e.g., visually or optically. In some cases, the volumes or thickness of a layer may be estimated statistically, e.g., by determining the amount of fluid or material present in a microparticle, and assuming that the microparticle is spherical, calculating the volume and/or thicknesses of the fluid around the microparticle.

In addition, in some embodiments, the thickness may be determined as a percentage of the diameter of the overall microparticle. For example, the thickness of the second layer (or other inner layer having a relatively thin thickness) within the microparticle may be than about 20%, less than about 15%, less than about 10%, less than about 5%, less than about 3%, less than about 1%, less than about 0.5%, less than about 0.3%, or less than about 0.1% of the diameter of the overall microparticle.

In addition, in some embodiments, the second layer (or other inner layer having a relatively thin shell) may comprise a relatively small percentage by volume of the overall microparticle. For example, the second layer may comprise less than about 20%, less than about 15%, less than about 10%, less than about 5%, less than about 3%, less than about 1%, less than about 0.5%, less than about 0.3%, or less than about 0.1% of the overall microparticle. In another set of embodiments, the second layer (or other inner layer having a relatively thin shell) may have a thickness such that the difference between the average diameter of a microparticle containing the second layer and the average diameter of an interior portion contained therein is less than about 20% of the average diameter of the overall microparticle, and in some cases, less than about 15%, less than about 10%, less than about 5%, less than about 3%, less than about 1%, less than about 0.5%, less than about 0.3%, or less than about 0.1% of the average diameter of the overall microparticle.

In some embodiments, the second layer (or other inner layer having a relatively thin shell) may have an average thickness of less than about 0.05, less than about 0.01, less than about 0.005, or less than about 0.001 times the average cross-sectional diameter of the microparticle, or between about 0.0005 and about 0.05, between about 0.0005 and about 0.01, between about 0.0005 and about 0.005, or between about 0.0005 and about 0.001 times the average cross-sectional diameter of the microparticle. In some embodiments, the second layer (or other inner layer having a relatively thin shell) of a microparticle may have an average thickness of less than about 1 micron, less than about 500 nm, or less than about 100 nm, or between about 50 nm and about 1 micron, between about 50 nm and about 500 nm, or between about 50 nm and about 100 nm. One of ordinary skill in the art would be capable of determining the average thickness, for example, by examining scanning electron microscope (SEM) images of the microparticles.

Some aspects of the present invention are generally directed to systems and methods for forming such droplets. In one set of embodiments, for example, various microfluidic conduits can be positioned to create the multiple emulsion droplets, e.g., in series. In some cases, e.g., by controlling the flow of a fluid through a conduit, surprisingly thin inner layers of fluid may be created. In some cases, these may be rigidified as discussed herein.

In one set of embodiments, a first conduit may be used to inject a first fluid into a second conduit containing a second fluid, which may be immiscible with the first fluid. In some cases, relatively low flow rates of the first fluid can be used, i.e., relative to the second fluid, e.g., under “dripping” conditions. The first fluid thus may form relatively large droplets of first fluid contained within the second fluid.

In some embodiments, the first conduit may have a cross-sectional dimension of less than about 1 mm, less than about 500 micrometers, less than about 200 micrometers, less than about 100 micrometers, less than about 75 micrometers, less than about 50 micrometers, or other dimensions as discussed herein. The cross-sectional area of the first conduit may be substantially constant, or may vary. For instance, the first conduit may be tapered. In certain embodiments, the first conduit is substantially smaller than the second conduit at the point where the first conduit opens into the second conduit. For instance, the first conduit may have a cross-sectional area of the exit opening that is no more than about 75%, no more than about 50%, no more than about 45%, no more than about 40%, no more than about 35%, no more than about 30%, no more than about 25%, no more than about 20%, no more than about 15%, no more than about 10%, or no more than about 5% of the cross-sectional area of the second conduit at that location.

In some embodiments, the first fluid droplets, being relatively discrete may not completely fill the second conduit; thus, the balance of the second conduit may be filled with the second fluid. Accordingly, the amount of first fluid within the channel, relative to the second fluid, may be increased. In one set of embodiments, this may be performed by using an exit opening that is smaller than the droplets of the first fluid, e.g., the average diameter of the exit opening may be smaller than the average diameter of the droplets of first fluid as they are created within the second conduit. Without wishing to be bound by any theory, it is believed that such a constriction allows for the exiting fluid to be mostly the first fluid, thereby allowing relatively larger first droplets contained in relatively smaller second droplets to be created, i.e., the second droplets would form a thin “shell” or have a relatively thin thickness surrounding the inner, first droplets.

The second conduit may gradually or suddenly reach the diameter of the exit opening. In one set of embodiments, a tapered region may be used. The length of the tapered region may be any suitable length as determined in the direction of average fluid flow within the channel; for example, the length can be less than about 1 mm, less than about 500 micrometers, less than about 300 micrometers, less than about 100 micrometers, less than about 50 micrometers, less than about 30 micrometers, less than about 10 micrometers, etc.

In some embodiments, the second conduit may have a cross-sectional dimension of less than about 1 mm, less than about 500 micrometers, less than about 200 micrometers, less than about 100 micrometers, less than about 75 micrometers, less than about 50 micrometers, or other dimensions as discussed herein. In some cases, the cross-sectional area of the second conduit may vary. In some cases, the second conduit is substantially smaller than the second conduit at the point where the second conduit opens into the third conduit. For instance, the second conduit may have a cross-sectional area of the exit opening that is no more than about 75%, no more than about 50%, no more than about 45%, no more than about 40%, no more than about 35%, no more than about 30%, no more than about 25%, no more than about 20%, no more than about 15%, no more than about 10%, or no more than about 5% of the cross-sectional area of the third conduit at that location.

In some cases, the fluids and the walls may be chosen such that the second fluid is preferentially attracted to the walls, relative to the first fluid. This may be inherently determined by the fluids and the material forming the walls, and/or the walls may be treated in some fashion to render them more attractive to the second fluid, relative to the first fluid. Examples of treating the walls, e.g., with a sol-gel coating, to control their hydrophilicity and/or hydrophobicity, and/or their attraction to the second fluid relative to the first fluid, are discussed in more detail herein. Without wishing to be bound by any theory, it is believed that under such conditions, the tapering of the conduits causes the first fluid to form elongated droplets that substantially fill the exit opening of the second conduit; however, due to the attraction of the second fluid to the walls of the conduit, relative to the first fluid, a thin stream of second fluid remains along with the first fluid as the fluids pass through the exit opening of the second conduit. In such a fashion, a thin second fluidic shell may be created around the first fluid droplet. Furthermore, if the flow rates of the first fluid and/or the second fluid are kept relatively low, e.g., under “dripping” conditions, the amount of second fluid exiting through the exit opening may be relatively small, e.g., only a small volume of second fluid passes through the exit opening, relative to the first fluid, which can further result in a relatively small shell of second fluid surrounding the droplet of first fluid.

Upon exiting the exit opening of the second conduit, the first fluid (surrounded by the second fluid) may encounter a third fluid and a fourth fluid contained within a third conduit. The third fluid may be immiscible with the second fluid and/or the fourth fluid, in some cases. Thus, the third fluid may be caused to form droplets surrounding the second fluid (and in turn, the first fluid) contained within the fourth fluid.

In some embodiments, the third conduit may have a cross-sectional dimension of less than about 1 mm, less than about 500 micrometers, less than about 200 micrometers, less than about 100 micrometers, less than about 75 micrometers, less than about 50 micrometers, or other dimensions as discussed herein. The cross-sectional area of the third conduit may be substantially constant, or may vary. For instance, the third conduit may be tapered.

The droplets of third fluid may then exit the third conduit through an entrance opening of a fourth conduit, e.g., for subsequent use. For example, one or more of the fluids may be hardened as discussed below to form a particle. The particle may have the same dimensions as the droplet prior to hardening.

In some embodiments, the fourth conduit may have a cross-sectional dimension of less than about 1 mm, less than about 500 micrometers, less than about 200 micrometers, less than about 100 micrometers, less than about 75 micrometers, less than about 50 micrometers, or other dimensions as discussed herein. The cross-sectional area of the fourth conduit may be substantially constant, or may vary. For instance, the fourth conduit may be tapered. In some cases, the fourth conduit is substantially smaller than the third conduit at the entrance opening to the fourth conduit. For instance, the fourth conduit may have a cross-sectional area of the exit opening that is no more than about 75%, no more than about 50%, no more than about 45%, no more than about 40%, no more than about 35%, no more than about 30%, no more than about 25%, no more than about 20%, no more than about 15%, no more than about 10%, or no more than about 5% of the cross-sectional area of the third conduit at that location. In some cases, the fourth conduit may have a diameter that changes moving away from the entrance opening, although in other cases, the diameter of the fourth conduit may be substantially constant.

In some aspects, multiple emulsion droplets can be formed that can include lipids (e.g., as in a liposome) and/or polymers (e.g., as in a polymersome). See, e.g., Int. Pat. Apl. Pub. Nos. WO 2009/148598 or WO 2006/096571, each incorporated herein by reference. Droplets such as polymersomes or liposomes may be formed, for example, using multiple emulsion techniques such as those described herein. Non-limiting examples of polymers that can be used include normal butyl acrylate and acrylic acid, which can be polymerized to form a copolymer of poly(normal-butyl acrylate)-poly(acrylic acid); poly(ethylene glycol) and poly(lactic acid), which can be polymerized to form a copolymer of poly(ethylene glycol)-poly(lactic acid); or poly(ethylene glycol) and poly(glycolic acid), which can be polymerized to form a copolymer of poly(ethylene glycol)-poly(glycolic acid). In some cases, the copolymer may comprise more than two types of monomers, for example, as in a copolymer of poly(ethylene glycol)-poly(lactic acid)-poly(glycolic acid). In some cases, the copolymer may include amphiphilic molecules. In some cases, the amphiphilic molecules can be lipids. The monomers may be distributed in any suitable order within the copolymer, for example, as separate blocks (e.g., a multiblock copolymer), randomly, alternating, etc. A polymer may include polymeric compounds, as well as compounds and species that can form polymeric compounds, such as prepolymers. Prepolymers include, for example, monomers and oligomers. In some cases, however, only polymeric compounds are used and prepolymers may not be appropriate.

In another aspect, the present invention can be used to produce polymersomes. In one set of embodiments, the polymersome is an asymmetric polymersome. In some cases, the polymersome comprises a multiblock copolymer. In some cases, at least one of the blocks of the copolymer is a biodegradable polymer. In one set of embodiments, a polymer within the polymersome comprises a copolymer, e.g., a block copolymer. The polymer may be, for instance, diblock or a triblock copolymer, which can be amphiphilic; examples of such polymers are discussed below. In some cases, where block copolymers, homopolymers may also be used (e.g., having the same composition as one of the blocks of the copolymer), e.g., to stabilize the vesicle. A “block copolymer” is given its usual definition in the field of polymer chemistry. A block is typically a portion of a polymer comprising a series of repeat units that are distinguishable from adjacent portions of the block. Thus, for instance, a diblock copolymer comprises a first repeat unit and a second repeat unit; a triblock copolymer includes a first repeat unit, a second repeat unit, and a third repeat unit; a multiblock copolymer includes a plurality of such repeat units, etc. As a specific example, a diblock copolymer may comprise a first portion defined by a first repeat unit and a second portion defined by a second repeat unit; in some cases, the diblock copolymer may further comprise a third portion defined by the first repeat unit (e.g,. arranged such that the first and third portions are separated by the second portion), and/or additional portions defined by the first and second repeat units.

Examples of biodegradable or biocompatible polymers include, but are not limited to, poly(lactic acid), poly(glycolic acid), polyanhydride, poly(caprolactone), poly(ethylene oxide), polybutylene terephthalate, starch, cellulose, chitosan, and/or combinations of these. A “biodegradable material,” as used herein, is a material that will degrade in the presence of physiological solutions (which can be mimicked using phosphate-buffered saline) on the time scale of days, weeks, or months (i.e., its half-life of degradation can be measured on such time scales). As used herein, “biocompatible” is given its ordinary meaning in the art. For instance, a biocompatible material may be one that is suitable for implantation into a subject without adverse consequences, for example, without substantial acute or chronic inflammatory response and/or acute rejection of the material by the immune system, for instance, via a T-cell response. It will be recognized, of course, that “biocompatibility” is a relative term, and some degree of inflammatory and/or immune response is to be expected even for materials that are highly biocompatible. However, non-biocompatible materials are typically those materials that are highly inflammatory and/or are acutely rejected by the immune system, i.e., a non-biocompatible material implanted into a subject may provoke an immune response in the subject that is severe enough such that the rejection of the material by the immune system cannot be adequately controlled, in some cases even with the use of immunosuppressant drugs, and often can be of a degree such that the material must be removed from the subject. In some cases, even if the material is not removed, the immune response by the subject is of such a degree that the material ceases to function; for example, the inflammatory and/or the immune response of the subject may create a fibrous “capsule” surrounding the material that effectively isolates it from the rest of the subject's body; materials eliciting such a reaction would also not be considered as “biocompatible.”

In some cases, a droplet, such as a triple or other multiple emulsion droplet, may include amphiphilic species such as amphiphilic polymers or lipids. The amphiphilic species typically includes a relatively hydrophilic portion, and a relatively hydrophobic portion. For instance, the hydrophilic portion may be a portion of the molecule that is charged, and the hydrophobic portion of the molecule may be a portion of the molecule that comprises hydrocarbon chains. Other amphiphilic species may also be used, besides diblock copolymers. For example, other polymers, or other species such as lipids or phospholipids may be used with the present invention.

In one set of embodiments, a liposome or a polymersome may be formed by removing a portion of the middle fluid of a multiple emulsion. For instance, a component of the middle fluid, such as a solvent or carrier, can be removed from the fluid, in part or in whole, through evaporation or diffusion. As an example, in some cases, the middle fluid comprises a solvent system used as a carrier, and dissolved or suspended polymers or lipids. After formation of a multiple emulsion, the solvent can be removed from the middle fluid using techniques such as evaporation or diffusion, leaving the polymers or lipids behind.

In another set of embodiments, however, a liposome or a polymersome may be formed by creating a triple or other multiple emulsion droplet having a relatively thin layer or shell or fluid, e.g., using techniques such as those described herein. For instance, the droplet may initially be created with a relatively thin layer or shell or fluid, and/or a portion of the fluid may be removed.

In addition, in some aspects of the invention, at least a portion of a triple or other multiple emulsion droplet may be solidified to form a particle or a capsule, for example, containing an inner fluid and/or a species as discussed herein. A fluid, e.g., within an outermost layer of a multiple emulsion droplet, can be solidified using any suitable method. For example, in some embodiments, the fluid may be dried, gelled, and/or polymerized, and/or otherwise solidified, e.g., to form a solid, or at least a semi-solid. The solid that is formed may be rigid in some embodiments, although in other cases, the solid may be elastic, rubbery, deformable, etc. In some cases, for example, an outermost layer of fluid may be solidified to form a solid shell at least partially containing an interior containing a fluid and/or a species. Any technique able to solidify at least a portion of a fluidic droplet can be used. For example, in some embodiments, a fluid within a fluidic droplet may be removed to leave behind a material (e.g., a polymer) capable of forming a solid shell. In other embodiments, a fluidic droplet may be cooled to a temperature below the melting point or glass transition temperature of a fluid within the fluidic droplet, a chemical reaction may be induced that causes at least a portion of the fluidic droplet to solidify (for example, a polymerization reaction, a reaction between two fluids that produces a solid product, etc.), or the like. Other examples include pH-responsive or molecular-recognizable polymers, e.g., materials that gel upon exposure to a certain pH, or to a certain species. In some embodiments, a fluidic droplet is solidified by increasing the temperature of the fluidic droplet. For instance, a rise in temperature may drive out a material from the fluidic droplet (e.g., within the outermost layer of a multiple emulsion droplet) and leave behind another material that forms a solid. Thus, in some cases, an outermost layer of a multiple emulsion droplet may be solidified to form a solid shell that encapsulates one or more fluids and/or species.

In addition, in one set of embodiments, the second droplet or fluid may rigidified, e.g., to form a polymer or a gel (e.g., in addition to or instead of rigidifying or the outer fluid as discussed above). Thus, a second fluid may be caused to form a rigidified layer within the droplet. For example, in one set of embodiments, after the double emulsion droplet has been prepared, the fluid may be polymerized or gelled, for example, by applying ultraviolet light or a change in temperature. For instance, the fluid may contain a monomer that can be polymerized, or a polymer that may be induced to form a gel, e.g., upon reaction with an initiator (e.g., in the presence of ultraviolet light). As another example, the fluid may contain a temperature-sensitive gel which may be solidified with a suitable change in temperature. For instance, a droplet may contain temperature-sensitive agarose gel may formed at an elevated temperature (e.g., above room temperature, about 25° C.), then cooled (e.g., to room temperature or to a temperature below room temperature); or the droplet may be formed at room temperature, then cooled to a temperature below room temperature, or the like. Still other examples for rigidifying the second droplet include, but are not limited to pH-responsive or molecular-recognizable polymers and other reactions as discussed above.

In some embodiments, for instance, the fluid may be polymerized or gelled chemically. For example, a chemical reaction or a cross-linking reaction may be induced in a fluid to cause polymerization or gelation to occur. In some embodiments, the polymerization reaction is a free-radical polymerization reaction, e.g., which may be initiated by exposing suitable reactants to heat and/or light, such as ultraviolet (UV) light, and/or an initiator, such as a photoinitiator able to produce free radicals (e.g., via molecular cleavage) upon exposure to light. Examples include, but are not limited to, Irgacure 2559, 2-hydroxy-4-(3-triethoxysilylpropoxy)-diphenylketone, or tetramethylethylenediamine. Examples of suitable polymers include, but are not limited to polyacrylamide, poly(N-isopropylacrylamide), or poly(ethylene glycol diacrylate). Other species may be present as well.

According to certain aspects, the systems and methods described herein can be used in a plurality of applications. For example, fields in which the particles and multiple emulsions described herein may be useful include, but are not limited to, food, beverage, health and beauty aids, paints and coatings, chemical separations, agricultural applications, and drugs and drug delivery. For instance, a precise quantity of a fluid, drug, pharmaceutical, or other species can be contained in a droplet or particle designed to release its contents under particular conditions. In some instances, cells can be contained within a droplet or particle, and the cells can be stored and/or delivered, e.g., to a target medium, for example, within a subject. Other species that can be contained within a droplet or particle and delivered to a target medium include, for example, biochemical species such as nucleic acids such as siRNA, RNAi and DNA, proteins, peptides, or enzymes. Additional species that can be contained within a droplet or particle include, but are not limited to, colloidal particles, magnetic particles, nanoparticles, quantum dots, fragrances, proteins, indicators, dyes, fluorescent species, chemicals, or the like. The target medium may be any suitable medium, for example, water, saline, an aqueous medium, a hydrophobic medium, or the like.

In one particular set of embodiments, particles (including capsules) comprising relatively thin shells can be formed using the multiple emulsion techniques described herein. In some cases, at least some of the particles may comprise a solid portion or shell at least partially containing an interior containing a fluid and/or a species. The shells of the particles can comprise a polymer in some embodiments. Examples include, but are not limited to, polystyrene, polycaprolactone, polyisoprene, poly(lactic acid), polystyrene (PS), polycaprolactone (PCL), polyisoprene (PIP), poly(lactic acid), polyethylene, polypropylene, polyacrylonitrile, polyimide, polyamide, and/or mixtures and/or co-polymers of these and/or other polymers. The carrying fluid may be used in some embodiments as a vehicle used to contact the particles with a target medium, and/or the carrying fluid may be substituted by a suitable vehicle, as discussed elsewhere herein. When the particles contact the target medium, at least a portion of the shells of the particles can be disrupted in some cases, for instance, such that at least some of the fluid and/or species within the particles is expelled or otherwise transported from the particles and into the target medium. Of course, it should be understood that the particles may be used in other applications as well, e.g., as discussed herein.

The particles or droplets described herein may have any suitable average cross-sectional diameter. Those of ordinary skill in the art will be able to determine the average cross-sectional diameter of a single and/or a plurality of particles or droplets, for example, using laser light scattering, microscopic examination, or other known techniques. The average cross-sectional diameter of a single particle or droplet, in a non-spherical particle or droplet, is the diameter of a perfect sphere having the same volume as the non-spherical particle or droplet. The average cross-sectional diameter of a particle or droplet (and/or of a plurality or series of particles or droplets) may be, for example, less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers, or between about 50 micrometers and about 1 mm, between about 10 micrometers and about 500 micrometers, or between about 50 micrometers and about 100 micrometers in some cases. The average cross-sectional diameter may also be at least about 1 micrometer, at least about 2 micrometers, at least about 3 micrometers, at least about 5 micrometers, at least about 10 micrometers, at least about 15 micrometers, or at least about 20 micrometers in certain cases. In some embodiments, at least about 50%, at least about 75%, at least about 90%, at least about 95%, or at least about 99% of the particles or droplets within a plurality of particles or droplets has an average cross-sectional diameter within any of the ranges outlined in this paragraph.

For many applications, it may be desirable to deliver a plurality of particles or droplets, at least some of which contain a fluid and/or a species such as those described herein, to a target. In order to ensure predictable delivery, some embodiments advantageously employ particles or droplets with relatively consistent properties. For example, in some embodiments, a plurality of particles or droplets is provided wherein the distribution of thicknesses of the outermost layer among the plurality of particles or droplets is relatively uniform. In some embodiments, a plurality of particles or droplets is provided having an overall thickness, measured as the average of the average thicknesses of each of the plurality of particles or droplets. In some cases, the distribution of the average thicknesses can be such that no more than about 5%, no more than about 2%, or no more than about 1% of the particles or droplets have an outermost layer with an average thickness thinner than 90% (or thinner than 95%, or thinner than 99%) of the overall average thickness and/or thicker than 110% (or thicker than 105%, or thicker than about 101%) of the overall average thickness of the outermost layer.

The plurality of particles or droplets may have relatively uniform cross-sectional diameters in certain embodiments. The use of particles or droplets with relatively uniform cross-sectional diameters can allow one to control viscosity, the amount of species delivered to a target, and/or other parameters of the delivery of fluid and/or species from the particles or droplets. In some embodiments, the particles or droplets of particles is monodisperse, or the plurality of particles or droplets has an overall average diameter and a distribution of diameters such that no more than about 5%, no more than about 2%, or no more than about 1% of the particles or droplets have a diameter less than about 90% (or less than about 95%, or less than about 99%) and/or greater than about 110% (or greater than about 105%, or greater than about 101%) of the overall average diameter of the plurality of particles or droplets.

In some embodiments, the plurality of particles or droplets has an overall average diameter and a distribution of diameters such that the coefficient of variation of the cross-sectional diameters of the particles or droplets is less than about 10%, less than about 5%, less than about 2%, between about 1% and about 10%, between about 1% and about 5%, or between about 1% and about 2%. The coefficient of variation can be determined by those of ordinary skill in the art, and may be defined as:

cᵥ=σ/|μ|

wherein σ is the standard deviation and μ is the mean.

In certain aspects of the present invention, as discussed, multiple emulsions are formed by flowing fluids through one or more channels. The system may be a microfluidic system. “Microfluidic,” as used herein, refers to a device, apparatus, or system including at least one fluid channel having a cross-sectional dimension of less than about 1 millimeter (mm), and in some cases, a ratio of length to largest cross-sectional dimension of at least 3:1. One or more channels of the system may be a capillary tube. In some cases, multiple channels are provided, and in some embodiments, at least some are nested, as described herein. The channels may be in the microfluidic size range and may have, for example, average inner diameters, or portions having an inner diameter, of less than about 1 millimeter, less than about 300 micrometers, less than about 100 micrometers, less than about 30 micrometers, less than about 10 micrometers, less than about 3 micrometers, or less than about 1 micrometer, thereby providing droplets having comparable average diameters. One or more of the channels may (but not necessarily), in cross-section, have a height that is substantially the same as a width at the same point. In cross-section, the channels may be rectangular or substantially non-rectangular, such as circular or elliptical.

As used herein, the term “fluid” generally refers to a substance that tends to flow and to conform to the outline of its container, i.e., a liquid, a gas, a viscoelastic fluid, etc. In one embodiment, the fluid is a liquid. Typically, fluids are materials that are unable to withstand a static shear stress, and when a shear stress is applied, the fluid experiences a continuing and permanent distortion. The fluid may have any suitable viscosity that permits flow. If two or more fluids are present, each fluid may be independently selected among essentially any fluids (liquids, gases, and the like) by those of ordinary skill in the art, by considering the relationship between the fluids.

A variety of materials and methods, according to certain aspects of the invention, can be used to form articles or components such as those described herein, e.g., channels such as microfluidic channels, chambers, etc. For example, various articles or components can be formed from solid materials, in which the channels can be formed via micromachining, film deposition processes such as spin coating and chemical vapor deposition, laser fabrication, photolithographic techniques, etching methods including wet chemical or plasma processes, 3D printing, and the like. See, for example, Scientific American, 248:44-55, 1983 (Angell, et al).

In one set of embodiments, various structures or components of the articles described herein can be formed from glass or a polymer, for example, an elastomeric polymer such as polydimethylsiloxane (“PDMS”), polytetrafluoroethylene (“PTFE” or Teflon®), epoxy, norland optical adhesive, or the like. For instance, according to one embodiment, microfluidic channels may be formed from glass tubes or capillaries. In addition, in some cases, a microfluidic channel may be implemented by fabricating the fluidic system separately using PDMS or other soft lithography techniques (details of soft lithography techniques suitable for this embodiment are discussed in the references entitled “Soft Lithography,” by Younan Xia and George M. Whitesides, published in the Annual Review of Material Science, 1998, Vol. 28, pages 153-184, and “Soft Lithography in Biology and Biochemistry,” by George M. Whitesides, Emanuele Ostuni, Shuichi Takayama, Xingyu Jiang and Donald E. Ingber, published in the Annual Review of Biomedical Engineering, 2001, Vol. 3, pages 335-373; each of these references is incorporated herein by reference). In addition, in some embodiments, various structures or components of the articles described herein can be formed of a metal, for example, stainless steel.

Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The device may also be formed from composite materials, for example, a composite of a polymer and a semiconductor material.

In some embodiments, various structures or components of the article are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, waxes, or mixtures or composites thereof heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, dodecyltrichlorosilanes, etc.

Silicone polymers are used in certain embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of various structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour, about 3 hours, about 12 hours, etc. Also, silicone polymers, such as PDMS, can be elastomeric and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures or channels from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, structures can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable or bonded to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy et al.), incorporated herein by reference.

Different components can be fabricated of different materials. For example, a base portion including a bottom wall and side walls can be fabricated from an opaque material such as silicon or PDMS, and a top portion can be fabricated from a transparent or at least partially transparent material, such as glass or a transparent polymer, for observation and/or control of the fluidic process. Components can be coated so as to expose a desired chemical functionality to fluids that contact interior channel walls, where the base supporting material does not have a precise, desired functionality. For example, components can be fabricated as illustrated, with interior channel walls coated with another material, e.g., as discussed herein. Material used to fabricate various components of the systems and devices of the invention, e.g., materials used to coat interior walls of fluid channels, may desirably be selected from among those materials that will not adversely affect or be affected by fluid flowing through the fluidic system, e.g., material(s) that is chemically inert in the presence of fluids to be used within the device. A non-limiting example of such a coating is disclosed below; additional examples are disclosed in Int. Pat. Apl. Ser. No. PCT/US2009/000850, filed Feb. 11, 2009, entitled “Surfaces, Including Microfluidic Channels, With Controlled Wetting Properties,” by Weitz, et al., published as WO 2009/120254 on Oct. 1, 2009, incorporated herein by reference.

In some embodiments, certain microfluidic structures of the invention (or interior, fluid-contacting surfaces) may be formed from certain oxidized silicone polymers. Such surfaces may be more hydrophilic than the surface of an elastomeric polymer. Such hydrophilic surfaces can thus be more easily filled and wetted with aqueous solutions.

In some embodiments, a bottom wall of a microfluidic device of the invention is formed of a material different from one or more side walls or a top wall, or other components. For example, in some embodiments, the interior surface of a bottom wall comprises the surface of a silicon wafer or microchip, or other substrate. Other components may, as described above, be sealed to such alternative substrates. Where it is desired to seal a component comprising a silicone polymer (e.g. PDMS) to a substrate (bottom wall) of different material, the substrate may be selected from the group of materials to which oxidized silicone polymer is able to irreversibly seal (e.g., glass, silicon, silicon oxide, quartz, silicon nitride, polyethylene, polystyrene, epoxy polymers, and glassy carbon surfaces which have been oxidized). Alternatively, other sealing techniques may be used, as would be apparent to those of ordinary skill in the art, including, but not limited to, the use of separate adhesives, bonding, solvent bonding, ultrasonic welding, etc.

Thus, in certain embodiments, the design and/or fabrication of the article may be relatively simple, e.g., by using relatively well-known soft lithography and other techniques such as those described herein. In addition, in some embodiments, rapid and/or customized design of the article is possible, for example, in terms of geometry. In one set of embodiments, the article may be produced to be disposable, for example, in embodiments where the article is used with substances that are radioactive, toxic, poisonous, reactive, biohazardous, etc., and/or where the profile of the substance (e.g., the toxicology profile, the radioactivity profile, etc.) is unknown. Another advantage to forming channels or other structures (or interior, fluid-contacting surfaces) from oxidized silicone polymers is that these surfaces can be much more hydrophilic than the surfaces of typical elastomeric polymers (where a hydrophilic interior surface is desired). Such hydrophilic channel surfaces can thus be more easily filled and wetted with aqueous solutions than can structures comprised of typical, unoxidized elastomeric polymers or other hydrophobic materials.

In one set of embodiments, one or more of the channels within the device may be relatively hydrophobic or relatively hydrophilic, e.g. inherently, and/or by treating one or more of the surfaces or walls of the channel to render them more hydrophobic or hydrophilic. Generally, the fluids that are formed droplets in the device are substantially immiscible, at least on the time scale of forming the droplets, and the fluids will often have different degrees of hydrophobicity or hydrophilicity. Thus, for example, a first fluid may be more hydrophilic (or more hydrophobic) relative to a second fluid, and the first and the second fluids may be substantially immiscible. Thus, the first fluid can from a discrete droplet within the second fluid, e.g., without substantial mixing of the first fluid and the second fluid (although some degree of mixing may nevertheless occur under some conditions). Similarly, the second fluid may be more hydrophilic (or more hydrophobic) relative to a third fluid (which may be the same or different than the first fluid), and the second and third fluids may be substantially immiscible.

Accordingly, in some cases, a surface of a channel may be relatively hydrophobic or hydrophilic, depending on the fluid contained within the channel. In one set of embodiments, a surface of the channel is hydrophobic or hydrophilic relative to other surfaces within the device. In addition, in some embodiments, a relatively hydrophobic surface may exhibit a water contact angle of greater than about 90°, and/or a relatively hydrophilic surface may exhibit a water contact angle of less than about 90°.

In some cases, relatively hydrophobic and/or hydrophilic surfaces may be used to facilitate the flow of fluids within the channel, e.g., to maintain the nesting of multiple fluids within the channel in a particular order.

In some aspects, as previously discussed, emulsions such as those described herein may be prepared by controlling the hydrophilicity and/or hydrophobicity of the channels used to form the emulsion. In one set of embodiments, the hydrophilicity and/or hydrophobicity of the channels may be controlled by coating a sol-gel onto at least a portion of a channel. For instance, in one embodiment, relatively hydrophilic and relatively hydrophobic portions may be created by applying a sol-gel to the channel surfaces, which renders them relatively hydrophobic. The sol-gel may comprise an initiator, such as a photoinitiator. Portions (e.g., channels, and/or portions of channels) may be rendered relatively hydrophilic by filling the channels with a solution containing a hydrophilic moiety (for example, acrylic acid), and exposing the portions to a suitable trigger for the initiator (for example, light or ultraviolet light in the case of a photoinitiator). For example, the portions may be exposed by using a mask to shield portions in which no reaction is desired, by directed a focused beam of light or heat onto the portions in which reaction is desired, or the like. In the exposed portions, the initiator may cause the reaction (e.g., polymerization) of the hydrophilic moiety to the sol-gel, thereby rendering those portions relatively hydrophilic (for instance, by causing poly(acrylic acid) to become grafted onto the surface of the sol-gel coating in the above example).

As is known to those of ordinary skill in the art, a sol-gel is a material that can be in a sol or a gel state, and typically includes polymers. The gel state typically contains a polymeric network containing a liquid phase, and can be produced from the sol state by removing solvent from the sol, e.g., via drying or heating techniques. In some cases, the sol may be pretreated before being used, for instance, by causing some polymerization to occur within the sol.

In some embodiments, the sol-gel coating may be chosen to have certain properties, for example, having a certain hydrophobicity. The properties of the coating may be controlled by controlling the composition of the sol-gel (for example, by using certain materials or polymers within the sol-gel), and/or by modifying the coating, for instance, by exposing the coating to a polymerization reaction to react a polymer to the sol-gel coating, as discussed below.

For example, the sol-gel coating may be made more hydrophobic by incorporating a hydrophobic polymer in the sol-gel. For instance, the sol-gel may contain one or more silanes, for example, a fluorosilane (i.e., a silane containing at least one fluorine atom) such as heptadecafluorosilane, or other silanes such as methyltriethoxy silane (MTES) or a silane containing one or more lipid chains, such as octadecylsilane or other CH₃(CH₂)ₙ— silanes, where n can be any suitable integer. For instance, n may be greater than 1, 5, or 10, and less than about 20, 25, or 30. The silanes may also optionally include other groups, such as alkoxide groups, for instance, octadecyltrimethoxysilane. In general, most silanes can be used in the sol-gel, with the particular silane being chosen on the basis of desired properties such as hydrophobicity. Other silanes (e.g., having shorter or longer chain lengths) may also be chosen in other embodiments of the invention, depending on factors such as the relative hydrophobicity or hydrophilicity desired. In some cases, the silanes may contain other groups, for example, groups such as amines, which would make the sol-gel more hydrophilic. Non-limiting examples include diamine silane, triamine silane, or N-(3-(trimethoxysilyl)propyl) ethylene diamine silane. The silanes may be reacted to form oligomers or polymers within the sol-gel, and the degree of polymerization (e.g., the lengths of the oligomers or polymers) may be controlled by controlling the reaction conditions, for example by controlling the temperature, amount of acid present, or the like. In some cases, more than one silane may be present in the sol-gel. For instance, the sol-gel may include fluorosilanes to cause the resulting sol-gel to exhibit greater hydrophobicity, and other silanes (or other compounds) that facilitate the production of polymers. In some cases, materials able to produce SiO₂ compounds to facilitate polymerization may be present, for example, TEOS (tetraethyl orthosilicate).

It should be understood that the sol-gel is not limited to containing only silanes, and other materials may be present in addition to, or in place of, the silanes. For instance, the coating may include one or more metal oxides, such as SiO₂, vanadia (V₂O₅), titania (TiO₂), and/or alumina (Al₂O₃).

In some instances, the microfluidic channel is present in a material suitable to receive the sol-gel, for example, glass, metal oxides, or polymers such as polydimethylsiloxane (PDMS) and other siloxane polymers. For example, in some cases, the microfluidic channel may be one in which contains silicon atoms, and in certain instances, the microfluidic channel may be chosen such that it contains silanol (Si—OH) groups, or can be modified to have silanol groups. For instance, the microfluidic channel may be exposed to an oxygen plasma, an oxidant, or a strong acid to cause the formation of silanol groups on the microfluidic channel.

The sol-gel may be present as a coating on the microfluidic channel, and the coating may have any suitable thickness. For instance, the coating may have a thickness of no more than about 100 micrometers, no more than about 30 micrometers, no more than about 10 micrometers, no more than about 3 micrometers, or no more than about 1 micrometer. Thicker coatings may be desirable in some cases, for instance, in applications in which higher chemical resistance is desired. However, thinner coatings may be desirable in other applications, for instance, within relatively small microfluidic channels.

In one set of embodiments, the hydrophobicity of the sol-gel coating can be controlled, for instance, such that a first portion of the sol-gel coating is relatively hydrophobic, and a second portion of the sol-gel coating is relatively hydrophilic. The hydrophobicity of the coating can be determined using techniques known to those of ordinary skill in the art, for example, using contact angle measurements such as those discussed herein. For instance, in some cases, a first portion of a microfluidic channel may have a hydrophobicity that favors an organic solvent to water, while a second portion may have a hydrophobicity that favors water to the organic solvent. In some cases, a hydrophilic surface is one that has a water contact angle of less than about 90° while a hydrophobic surface is one that has a water contact angle of greater than about 90°.

The hydrophobicity of the sol-gel coating can be modified, for instance, by exposing at least a portion of the sol-gel coating to a polymerization reaction to react a polymer to the sol-gel coating. The polymer reacted to the sol-gel coating may be any suitable polymer, and may be chosen to have certain hydrophobicity properties. For instance, the polymer may be chosen to be more hydrophobic or more hydrophilic than the microfluidic channel and/or the sol-gel coating. As an example, a hydrophilic polymer that could be used is poly(acrylic acid).

The polymer may be added to the sol-gel coating by supplying the polymer in monomeric (or oligomeric) form to the sol-gel coating (e.g., in solution), and causing a polymerization reaction to occur between the polymer and the sol-gel. For instance, free radical polymerization may be used to cause bonding of the polymer to the sol-gel coating. In some embodiments, a reaction such as free radical polymerization may be initiated by exposing the reactants to heat and/or light, such as ultraviolet (UV) light, optionally in the presence of a photoinitiator able to produce free radicals (e.g., via molecular cleavage) upon exposure to light. Those of ordinary skill in the art will be aware of many such photoinitiators, many of which are commercially available, such as Irgacur 2959 (Ciba Specialty Chemicals) or 2-hydroxy-4-(3-triethoxysilylpropoxy)-diphenylketone (SIH6200.0, ABCR GmbH & Co. KG).

The photoinitiator may be included with the polymer added to the sol-gel coating, or in some cases, the photoinitiator may be present within the sol-gel coating. For instance, a photoinitiator may be contained within the sol-gel coating, and activated upon exposure to light. The photoinitiator may also be conjugated or bonded to a component of the sol-gel coating, for example, to a silane. As an example, a photoinitiator such as Irgacur 2959 may be conjugated to a silane-isocyanate via a urethane bond, where a primary alcohol on the photoinitiator may participate in nucleophilic addition with the isocyanate group, which may produce a urethane bond.

It should be noted that only a portion of the sol-gel coating may be reacted with a polymer, in some embodiments of the invention. For instance, the monomer and/or the photoinitiator may be exposed to only a portion of the microfluidic channel, or the polymerization reaction may be initiated in only a portion of the microfluidic channel. As a particular example, a portion of the microfluidic channel may be exposed to light, while other portions are prevented from being exposed to light, for instance, by the use of masks or filters, or by using a focused beam of light. Accordingly, different portions of the microfluidic channel may exhibit different hydrophobicities, as polymerization does not occur everywhere on the microfluidic channel. As another example, the microfluidic channel may be exposed to UV light by projecting a de-magnified image of an exposure pattern onto the microfluidic channel. In some cases, small resolutions (e.g., 1 micrometer, or less) may be achieved by projection techniques.

Additional details of such coatings and other systems may be seen in U.S. Provisional Patent Application Ser. No. 61/040,442, filed Mar. 28, 2008, entitled “Surfaces, Including Microfluidic Channels, With Controlled Wetting Properties,” by Abate, et al.; and International Patent Application Serial No. PCT/US2009/000850, filed Feb. 11, 2009, entitled “Surfaces, Including Microfluidic Channels, With Controlled Wetting Properties,” by Abate, et al., each incorporated herein by reference.

Certain aspects of the invention are generally directed to techniques for scaling up or “numbering up” devices such as those discussed herein. For example, in some cases, relatively large numbers of devices may be used in parallel, for example at least about 10 devices, at least about 30 devices, at least about 50 devices, at least about 75 devices, at least about 100 devices, at least about 200 devices, at least about 300 devices, at least about 500 devices, at least about 750 devices, or at least about 1,000 devices or more may be operated in parallel. In some cases, an array of such devices may be formed by stacking the devices horizontally and/or vertically. The devices may be commonly controlled, or separately controlled, and can be provided with common or separate sources of various fluids, depending on the application.

Those of ordinary skill in the art will be aware of other techniques useful for scaling up or numbering up devices or articles such as those discussed herein. For example, in some embodiments, a fluid distributor can be used to distribute fluid from one or more inputs to a plurality of outputs, e.g., in one more devices. For instance, a plurality of articles may be connected in three dimensions. In some cases, channel dimensions are chosen that allow pressure variations within parallel devices to be substantially reduced. Other examples of suitable techniques include, but are not limited to, those disclosed in International Patent Application No. PCT/US2010/000753, filed Mar. 12, 2010, entitled “Scale-up of Microfluidic Devices,” by Romanowsky, et al., published as WO 2010/104597 on Nov. 16, 2010, incorporated herein by reference in its entirety.

The following documents are incorporated herein by reference in their entirety for all purposes: U.S. Provisional Application Ser. No. 61/980,541, filed Apr. 16, 2014, entitled “Systems and methods for producing droplet emulsions with relatively thin shells”; International Patent Publication Number WO 2004/091763, filed Apr. 9, 2004, entitled “Formation and Control of Fluidic Species,” by Link et al.; International Patent Publication Number WO 2004/002627, filed Jun. 3, 2003, entitled “Method and Apparatus for Fluid Dispersion,” by Stone et al.; International Patent Publication Number WO 2006/096571, filed Mar. 3, 2006, entitled “Method and Apparatus for Forming Multiple Emulsions,” by Weitz et al.; International Patent Publication Number WO 2005/021151, filed Aug. 27, 2004, entitled “Electronic Control of Fluidic Species,” by Link et al.; International Patent Publication Number WO 2008/121342, filed Mar. 28, 2008, entitled “Emulsions and Techniques for Formation,” by Chu et al.; International Patent Publication Number WO 2010/104604, filed Mar. 12, 2010, entitled “Method for the Controlled Creation of Emulsions, Including Multiple Emulsions,” by Weitz et al.; International Patent Publication Number WO 2011/028760, filed Sep. 1, 2010, entitled “Multiple Emulsions Created Using Junctions,” by Weitz et al.; International Patent Publication Number WO 2011/028764, filed Sep. 1, 2010, entitled “Multiple Emulsions Created Using Jetting and Other Techniques,” by Weitz et al.; International Patent Publication Number WO 2009/148598, filed Jun. 4, 2009, entitled “Polymersomes, Phospholipids, and Other Species Associated with Droplets,” by Shum, et al.; International Patent Publication Number WO 2011/116154, filed Mar. 16, 2011, entitled “Melt Emulsification,” by Shum, et al.; International Patent Publication Number WO 2009/148598, filed Jun. 4, 2009, entitled “Polymersomes, Colloidosomes, Liposomes, and other Species Associated with Fluidic Droplets,” by Shum, et al.; International Patent Publication Number WO 2012/162296, filed May 22, 2012, entitled “Control of Emulsions, Including Multiple Emulsions,” by Rotem, et al.; International Patent Publication Number WO 2013/006661, filed Jul. 5, 2012, entitled “Multiple Emulsions and Techniques for the Formation of Multiple Emulsions,” by Kim, et al.; and International Patent Publication Number WO 2013/032709, filed Aug. 15, 2012, entitled “Systems and Methods for Shell Encapsulation,” by Weitz, et al. Also incorporated herein by reference is U.S. Provisional Patent Application Ser. No. 62/083,721, filed Nov. 24, 2014, entitled “Systems and Methods for Encapsulation of Actives in Compartments or Sub-Compartments,” by Weitz, et al.

The following examples are intended to illustrate certain embodiments of the present invention, but do not exemplify the full scope of the invention.

### EXAMPLE 1

This example illustrates the encapsulation of a complex fluid (e.g., perfume in a hydrophobic solution) in polymer shells (shell materials). This example illustrates high encapsulation efficiency (e.g., over 90%), through use of polymer shells with ultra-thin water layer.

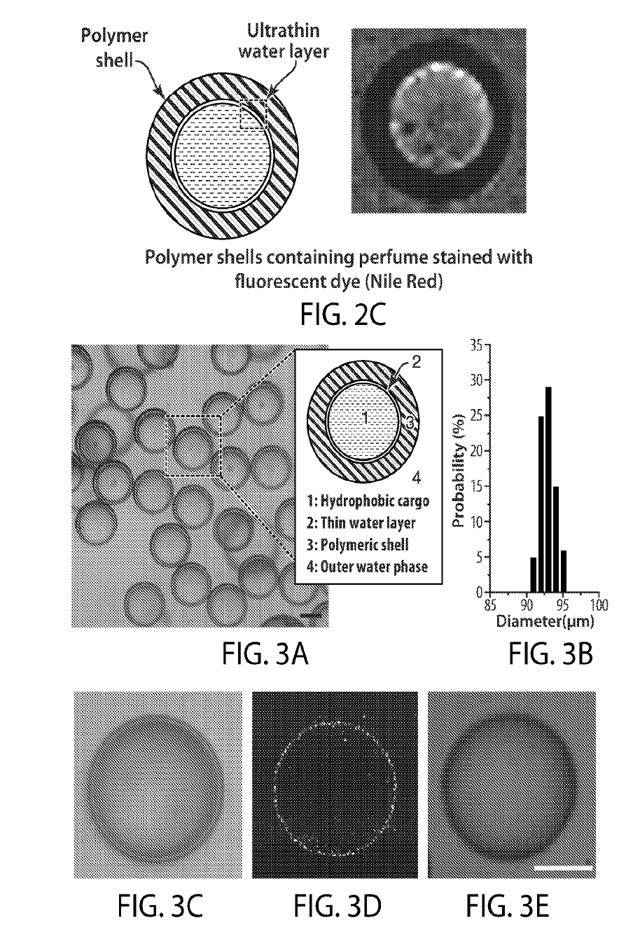

A biphasic flow is first created in a capillary device by forming a sheath flow of a thin water layer with high affinity to the glass capillary wall flowing along the inner wall of the capillary, surrounding the fluid containing perfume. A thin water layer facilitate sheath flow of the fluid containing the perfume and a hydrophobic monomer fluid, without mixing, which are simultaneously introduced into an orifice in the form of a coaxial flow, as is shown in FIG. 2A. This results in the formation of triple emulsions with a relatively thin water layer.

In this example, a solution containing perfume was used for the innermost phase, an aqueous solution with 1 wt % PVA was used for thin-water layer, ethoxylated trimethylolpropane triacrylate (ETPTA) was for the polymer shell, and an aqueous solution with 10% PVA as the continuous phase. Upon UV exposure, the perfume may be encapsulated within a polymeric shell.

This example shows high encapsulation efficiency (>90%) of perfume in polymer shell through use of triple emulsions with a relatively thin water layer.

### EXAMPLE 2

Emulsions have been widely used as carriers for food, drugs, and cosmetics due to their great capability of encapsulation. Recent advance in microfluidics enables precise control of multiphasic flows, leading to highly monodisperse emulsions with fine-tunable size, morphologies, and properties of each compartment. For example, double emulsion drops with an additional intermediate phase that separates the innermost drop from continuous phase can be generated, providing highly efficient encapsulation of hydrophilic or hydrophobic cargos while avoiding cross-contamination. See, e.g., U.S. Pat. Apl. Ser. No. 62/083,721, incorporated herein by reference. Additional flexibility can be achieved by consolidation of the intermediate phase in double emulsion drops to form polymeric microcapsules, vesicles, and colloidosomes; for instance, polymeric shell in microcapsules can be fine-tuned to facilitate controlled release to active cargo. Since many applications require the capsules to be dispersed in an aqueous phase, double emulsion-templated capsules may be used for encapsulation of hydrophilic cargo. With hydrophobic cargos protected by hydrophilic shells, the capsules can be re-dispersed into an aqueous phase.

This example shows triple emulsion drops with a thin water layer in a single step microfluidic emulsification, which are used achieve high encapsulation efficiency of hydrophobic cargo in polymeric microcapsules. A water-in-oil biphasic flow confined in an injection capillary co-flows with a hydrophobic photocurable oil phase, which are then emulsified by additional continuous aqueous phase, resulting in monodisperse triple emulsion drops with thin intermediate water layers in dripping mode. This allows encapsulation of hydrophobic cargo with high efficiency by minimizing the specific volume occupied by the thin water layer. Moreover, this example demonstrates that the thin water layer can be further tailored by adding hydrogel precursor; the polymerized hydrogel shell provides a physical barrier that separates the hydrophobic cargo from directly contacting the polymer shell. This hydrogel shell allows enhanced retention of a highly volatile small organic compound (alpha-pinene), since low molecular weight molecules have high mobility and thus quickly diffuse through the shell.

To make triple emulsion drops with the thin water layer, a glass capillary microfluidic device is used, comprised of two tapered circular capillaries inserted into a square capillary. 2-[methoxy(polyethyleneoxy)propyl]trimethoxysilane (PEG-silane) is used to make the circular capillary wall hydrophilic. In addition, a small tapered capillary is inserted into the injection capillary to facilitate simultaneous injection of two immiscible fluids. Another circular capillary is inserted into the square capillary at the other side to confine the flow near the injection tip, thereby increasing the flow velocity; this is also treated with PEG-silane to make the capillary wall hydrophilic, as schematically illustrated in FIG. 2A. FIG. 2A shows a microfluidic capillary device for production of oil-in-water-in-oil-in-water (o/w/o/w) triple emulsion drops with a thin intermediate water layer. FIG. 2B is an optical microscope image showing formation of the triple emulsions in collection capillary. Scale bar represents 50 micrometers.

An oil phase is injected through the small tapered capillary to form the innermost drop. An aqueous phase is injected through the injection capillary. The co-injection of these two immiscible fluids results in a coaxial biphasic flow of a thin water layer surrounding the innermost fluid of oil phase due to strong affinity of the aqueous phase to PEG-treated injection capillary. A photocurable solution is used for the second oil phase and injected through the interstices of the square and injection capillaries from the same side with the injection capillary. Additional aqueous phase is injected through the square capillary from the other side as the continuous phase. The coaxial biphasic jets from the injection capillary co-flow with the photocurable oil phase, which are emulsified by the continuous aqueous phase at the exit of the injection one, resulting in monodisperse triple emulsion drops with the thin water layer. These emulsion drops then flow downstream through the collection capillary, as shown in the optical image of FIG. 1B.

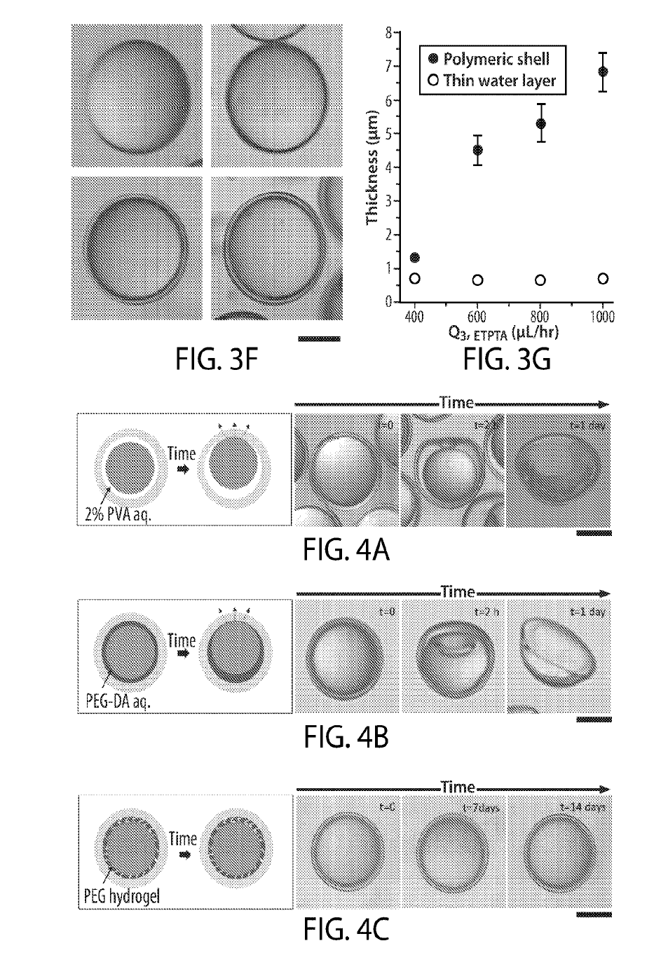

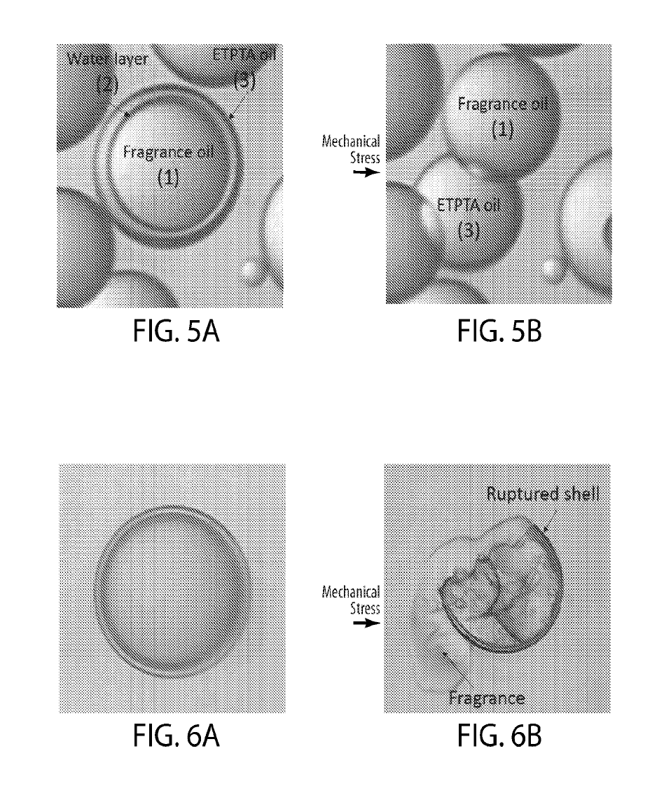

The thin water layer in the triple emulsion drop separates the innermost oil phase from the photocurable oil phase making up the shell, allowing encapsulation of hydrophobic cargo within a miscible hydrophobic shell. Furthermore, minimizing the volume occupied by this water layer increases loading capacity of the hydrophobic cargo within the emulsion drops. To validate this approach, a model fragrance was used (alpha-pinene, Sigma-Aldrich) for the innermost oil phase (Q1), an aqueous solution of 2% poly (vinyl alcohol) (PVA) for the thin water layer (Q2), photocurable ethoxylated trimethylolpropane triacrylate (ETPTA) as the second oil phase (Q3) and aqueous solution of 10% PVA as the continuous phase (Q4). To produce polymeric microcapsules, the stream of triple emulsion drops was exposed to UV illumination; this resulted in polymerization of the ETPTA monomer thereby forming a solid shell. The resulting microcapsules were monodisperse with a coefficient of variation of 2%, as shown in the size distribution curve in FIGS. 3A and 3B. To distinguish each layer within the microcapsules, the thin water layer was labeled with fluorescein sodium salt (green) and the innermost oil phase with Nile red (red). The microcapsules with a thin water layer separating the polymeric shell and the innermost oil phase, are shown in confocal images of FIGS. 3C-3E. Due to the optical resolution limit, it is impossible to measure the thickness of the thin water layer using image analysis. Instead, the triple emulsion drops were ruptured prior to the UV exposure to form two separated oil drops of the ETPTA and the innermost oil phase, as shown in bright-field images in FIG. 5. Then, the volume of innermost oil drop was measured and subtracted from the total volume of the triple emulsion drop. This gives us the volume initially occupied by the thin water layer in the triple emulsion drop. Based on this measurement, the thickness of the thin water layer was approximately 650 nm; therefore, the encapsulation efficiency of the hydrophobic cargo was estimated to be above 95%. FIG. 5 shows bright-field images showing rupturing of triple emulsion drop between two glass slides. This leads to formation of two separated oil drops of the ETPTA and the innermost oil phase (fragrance oil), while the water layer dissipates in a continuous aqueous media.

The polymeric shell thickness was tuned by adjusting the flow rate of the photocurable oil phase (Q3), as evidenced by capsules with different shell thicknesses in bright-field images of FIG. 3F. Importantly, while the thickness of the polymeric shell varies depending on the flow rate of Q3, that of the thin water layer was not affected by the flow rate of each phase, as shown in a plot of the shell thickness versus Q3 of FIG. 3G. This flow rate independence of the thickness of the water layer was attributed to the lubricated volume of water that preferentially wetted the inner wall of the injection capillary.

FIG. 3A shows a bright-field microscope image of monodisperse microcapsules by photopolymerization of the triple emulsion drops. FIG. 3B shows the size distribution of the triple emulsion drops. FIGS. 3C-3E show optical and confocal images of polymeric microcapsules incorporating two fluorescent dyes; fluorescein for the ultra-thin water layer and Nile red for the innermost oil phase. FIG. 3F shows bright-field images showing microcapsule with varying shell thickness, where Q3 is varied from 400 to 1000 microliters/h, and Q1, Q2 and Q4 are maintained at 1000, 1500, and 15000 microliters/h, respectively. FIG. 3G shows the size of the shell thickness as a function of flow rate of the photocurable oil phase (Q3), with a constant water layer thickness. Scale bars represent 50 microliters.

### EXAMPLE 3

While triple emulsion drops with the thin water layer allowed high encapsulation efficiency of the hydrophobic model fragrance, the density mismatch between the fragrance and the thin water layer (density of fragrance=0.858 and density of 2% PVA=1.01) resulted in the fragrance drop rising and directly contacting the inner surface of the polymeric shell; the hydrophobic fragrance imbibed into the hydrophobic polymer, leading to a rapid leakage and hence limiting the long-term storage. To achieve long-term storage of the fragrance, the formulation of the thin water layer was tailored by rigidifying the thin water layer, allowing it to act as a physical barrier and preventing direct exposure of the innermost fragrance with the polymeric shell. To demonstrate our strategy, triple emulsion drops were used with a water layer composed of an aqueous solution of 15% polyethylene glycol diacrylate (PEG-DA, Mn=700) with or without photoinitiator. In the presence of photoinitiator, PEG-DA precursor solution can be rapidly polymerized upon UV exposure, transforming into a thin hydrogel layer. To investigate the effect of a thin water layer composition in enhanced cargo retention, the capsules were dispersed into an aqueous solution and the leakage behavior of fragrance monitored using a bright-field microscope, as shown in bright-field images of FIG. 4.

As a control experiment, test microcapsules were tested with a thin layer composed of 2% PVA; these microcapsules lead to rapid leakage of fragrance through polymeric membrane. The subsequent volume loss of the fragrance causes the shell to buckle within 24 h, as shown in bright-field images of FIG. 4A (right of figure; schematic images on left). As expected, the fragrance drop surrounded by the thin water layer rose and directly contacted the polymeric membrane, resulting in rapid leakage of the fragrance through the membrane. However, by using triple emulsion drops with 15% PEG-DA with photoinitator, microcapsules were created with a thin hydrogel layer which is surrounded by the solidified polymeric shell, thereby forming a hydrophilic-hydrophobic hybrid shell. The resulting microcapsules with such hybrid polymeric shells notably improved the retention of fragrance, whereas a non-polymerized PEG-DA layer lead to a rapid release, as evidenced by buckled and intact polymeric shell in bright-field images of FIG. 4B and 4C, respectively. The leakage behavior from a non-polymerized PEG-DA layer is similar with that of the microcapsules with the thin layer of aqueous solution of 2% PVA. This result indicates that the thin hydrogel layer effectively prevented fragrance from being exposed to the polymer shell; thus the resulting hybrid shells enable long-term storage of the fragrance.

To test the long-term storage of fragrance in the microcapsules, mechanical stress was applied to rupture the polymeric capsules between two glass slides. A bright-field image exhibited the trace of fragrances released from the cracked polymeric shells, as shown in the bright-field image of FIG. 6. Although the hydrogel layer is very thin, it effectively suppressed the leakage of fragrance by separating them from the polymeric shell. FIG. 6 shows bright field images showing release of model fragrance encapsulated within a polymeric microcapsule by applying mechanical stress.

Here, monodisperse triple emulsion drops were produced with a thin water layer through a one-step microfluidic emulsification. Using this triple emulsion approach, high encapsulation efficiency (˜95%) was achieved of hydrophobic cargo within a hydrophobic polymeric shell, which is difficult to achieve by conventional emulsification techniques. The thickness of the polymeric shell could be controlled by adjusting the flow rate of the photocurable oil phase while keeping the thickness of the thin water layer constant; this approach allowed consistent production of the microcapsules with high encapsulation efficiency. Furthermore, the thin water layer can be formulated to rigidify into a cross-linked hydrogel, preventing direct exposure of the innermost oil drop from the shell while forming a hydrophilic-hydrophobic hybrid polymeric shell; thus this hybrid shell confers efficient diffusion barrier, allowing long-term storage of volatile hydrophobic cargo. The thin layer of the triple emulsion drops could be further tailored into omniphobic perfluorinated oil, facilitating encapsulation of hydrophilic or hydrophobic cargos, and even organic solvent. This approach should be well-suited for encapsulation of small active molecules such as fragrances, drugs, and nutrients.

FIG. 4 shows a series of bright-field microscope images showing the leakage of hydrophobic model fragrance (alpha-pinene) encapsulated in polymeric microcapsules, which is composed of (FIG. 4A) 2% PVA aqueous solution, (FIG. 4B) 15% PEG-DA aqueous solution, and (FIG. 4C) PEG cross-linked hydrogel. The leakage behavior is monitored in an aqueous solution. Scale bar represents 50 micrometers.

### EXAMPLE 4

Following are materials and methods used in the above examples.

To produce triple emulsion drops, alpha-pinene is injected as a model fragrance through the small tapered capillary with a typical flow rate of 1000 microliters/h. A solution of 2% aqueous solution of PVA (MW=13-23 kDa, Sigma-Aldrich) is simultaneously supplied through the injection capillary with a typical flow rate of 1000 microliters/h. In addition, ETPTA (Aldrich) containing 0.5 wt % photoinitiator (2-hydroxy-2-methylpropiophenone, Aldrich) is injected through the interstices of the square and injection with a typical volumetric flow rate of 1000 microliters/h. A 10 wt % aqueous solution of PVA is injected through the interstices of the square and collection capillary with a typical volumetric flow rate of 15000 microliters/h. Photopolymerization is achieved by UV exposure for 2 seconds (Omnicure S1000).

Preparation of microfluidic device and drop generation. A glass capillary microfluidic device is used to produce triple emulsion droplets with an ultra-thin middle layer. An injection capillary is prepared by tapering a 560 micrometer inner diameter cylindrical glass capillary (1B100-6, World Precision Instruments, Inc.) to 40 micrometer inner diameter; to make the inner wall hydrophobic, it is dipped into 2-[methoxy(polyethyleneoxy)propyl] trimethoxy silane (Gelest, Inc.) for 10 minutes and subsequently washed with DI water. The injection capillary is inserted into a square capillary (AIT Glass) whose inner width (1.05 mm) is slightly larger than that of the outer diameter of the injection capillary (1 mm). Next, a small tapered glass capillary is prepared (10 micrometer inner diameter) by heating and pulling a cylindrical capillary by hand using a gas torch; this capillary is inserted into the injection capillary for simultaneous injection of two immiscible fluids. Finally, a cylindrical collection capillary is inserted into the square capillary from the other end; this collection capillary is treated with 2-[methoxy(polyethyleneoxy)propyl] trimethoxy silane (Gelest, Inc.) to make the capillary wall hydrophilic. During drop generation, the volumetric flow rate was controlled by syringe pumps (Harvard Apparatus) and the production of emulsion drops was observed using an inverted microscope equipped with a high-speed camera (Phantom V9.0).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
a first droplet comprising a first fluid, the first droplet contained within a second layer comprising a gel, the gel contained within a third droplet comprising a third fluid,
wherein the gel has an average thickness, between the first droplet and the third droplet, of less than about 1000 nm.

2. The composition of claim 1, wherein the gel is a hydrogel.

3. The composition of any one of claim 1 or 2, wherein the gel comprises polyethylene glycol diacrylate.

4. The composition of any one of claims 1-3, wherein the gel has an average thickness of less than about 700 nm.

5. The composition of any one of claims 1-4, wherein the gel has an average thickness of less than about 500 nm.

6. The composition of any one of claims 1-5, wherein the first fluid and the third fluid are miscible.

7. The composition of any one of claims 1-6, wherein the first fluid and the third fluid are substantially identical.

8. The composition of any one of claims 1-7, wherein the first fluid is hydrophilic.

9. The composition of any one of claims 1-8, wherein the first fluid is hydrophobic.

10. The composition of any one of claims 1-9, wherein the first fluid and the third fluid are immiscible.

11. The composition of any one of claims 1-10, wherein the third droplet has an average diameter of less than about 1 micrometer.

12. The composition of any one of claims 1-11, wherein the gel has an average diameter of less than about 1 micrometer.

13. The composition of any one of claims 1-12, wherein the first droplet has an average diameter of less than about 1 micrometer.

14. The composition of any one of claims 1-13, wherein the gel comprises less than about 10% of the volume of the third droplet, and the first fluid comprises at least about 50% of the volume of the third droplet.

15. The composition of any one of claims 1-14, wherein the difference between the average diameter of the gel and the average diameter of the first droplet is less than about 10% of the average diameter of the third droplet.

16. The composition of any one of claims 1-15, wherein the first fluid and the third fluid are not in direct physical contact.

17. The composition of any one of claims 1-16, wherein the third droplet is contained within a carrying fluid.

18. The composition of claim 17, wherein the carrying fluid is immiscible with the third fluid.

19. The composition of any one of claims 1-18, wherein the third droplet is one of a plurality of multiple emulsion droplets, wherein the plurality of multiple emulsion droplets has a distribution of diameters such that at least 90% of the multiple emulsion droplets have a diameter greater than 90% and less than 110% of the average diameter of the plurality of multiple emulsion droplets.

20. A composition, comprising:
a first droplet comprising a first fluid, the first droplet contained within a second layer comprising a gel, the gel contained within a third droplet comprising a third fluid,
wherein the gel comprises less than about 10% of the volume of the third droplet, and the first fluid comprises at least about 50% of the volume of the third droplet.

21. The composition of claim 20, wherein the gel is a hydrogel.

22. The composition of any one of claim 20 or 21, wherein the gel comprises polyethylene glycol diacrylate.

23. The composition of any one of claims 20-22, wherein the gel comprises less than about 5% of the volume of the third droplet.

24. The composition of any one of claims 20-23, wherein the first fluid comprises at least about 70% of the volume of the third droplet.

25. The composition of any one of claims 20-24, wherein the first fluid comprises less than 80% of the volume of the third droplet.

26. The composition of any one of claims 20-25, wherein the gel has an average thickness, between the first droplet and the third droplet, of less than about 1000 nm.

27. The composition of claim 26, wherein the gel has an average thickness of less than about 700 nm.

28. The composition of any one of claim 26 or 27, wherein the gel has an average thickness of less than about 500 nm.

29. The composition of any one of claims 20-28, wherein the first fluid and the third fluid are miscible.

30. The composition of any one of claims 20-29, wherein the first fluid and the third fluid are substantially identical.

31. The composition of any one of claims 20-30, wherein the first fluid is hydrophilic.

32. The composition of any one of claims 20-31, wherein the first fluid is hydrophobic.

33. The composition of any one of claims 20-32, wherein the third droplet has an average diameter of less than about 1 micrometer.

34. The composition of any one of claims 20-33, wherein the gel has an average diameter of less than about 1 micrometer.

35. The composition of any one of claims 20-34, wherein the first droplet has an average diameter of less than about 1 micrometer.

36. The composition of any one of claims 20-35, wherein the difference between the average diameter of the gel and the average diameter of the first droplet is less than about 10% of the average diameter of the third droplet.

37. The composition of any one of claims 20-36, wherein the first fluid and the third fluid are not in direct physical contact.

38. The composition of any one of claims 20-37, wherein the third droplet is contained within a carrying fluid.

39. The composition of claim 38, wherein the carrying fluid is immiscible with the third fluid.

40. The composition of any one of claims 20-39, wherein the third droplet is one of a plurality of triple emulsion droplets, wherein the plurality of triple emulsion droplets has a distribution of diameters such that at least 90% of the triple emulsion droplets have a diameter greater than 90% and less than 110% of the average diameter of the plurality of triple emulsion droplets.

41. A composition, comprising:
a first droplet comprising a first fluid, the inner droplet contained within a second layer comprising a gel, the gel contained within a third droplet comprising a second fluid,
wherein the difference between the average diameter of the gel and the average diameter of the first droplet is less than about 10% of the average diameter of the third droplet.

42. The composition of claim 41, wherein the gel is a hydrogel.

43. The composition of any one of claim 41 or 42, wherein the gel comprises polyethylene glycol diacrylate.

44. The composition of any one of claims 41-43, wherein the difference between the average diameter of the gel and the average diameter of the first droplet is less than about 5% of the average diameter of the third droplet.

45. The composition of any one of claims 41-44, wherein the gel comprises less than about 5% of the volume of the third droplet.

46. The composition of any one of claims 41-45, wherein the gel has an average thickness, between the first droplet and the third droplet, of less than about 1000 nm.

47. The composition of claim 46, wherein the gel has an average thickness of less than about 700 nm.

48. The composition of any one of claim 46 or 47, wherein the gel has an average thickness of less than about 500 nm.

49. The composition of any one of claims 41-48, wherein the first fluid and the third fluid are miscible.

50. The composition of any one of claims 41-49, wherein the first fluid and the third fluid are substantially identical.

51. The composition of any one of claims 41-50, wherein the first fluid is hydrophilic.

52. The composition of any one of claims 41-51, wherein the first fluid is hydrophobic.

53. The composition of any one of claims 41-52, wherein the first fluid and the third fluid are mutually immiscible.

54. The composition of any one of claims 41-53, wherein the third droplet has an average diameter of less than about 1 micrometer.

55. The composition of any one of claims 41-54, wherein the gel has an average diameter of less than about 1 micrometer.

56. The composition of any one of claims 41-55, wherein the first droplet has an average diameter of less than about 1 micrometer.

57. The composition of any one of claims 41-56, wherein the gel comprises less than about 10% of the volume of the third droplet, and the first fluid comprises at least about 50% of the volume of the third droplet.

58. The composition of any one of claims 41-57, wherein the first fluid and the third fluid are not in direct physical contact.

59. The composition of any one of claims 41-58, wherein the third droplet is contained within a carrying fluid.

60. The composition of claim 59, wherein the carrying fluid is immiscible with the third fluid.

61. The composition of any one of claims 41-60, wherein the third droplet is one of a plurality of triple emulsion droplets, wherein the plurality of triple emulsion droplets has a distribution of diameters such that at least 90% of the triple emulsion droplets have a diameter greater than 90% and less than 110% of the average diameter of the plurality of triple emulsion droplets.
